(19) United States
(12) Patent Application Publication
(10) Pub. No.: US 2014/0142113 A1
(43) Pub. Date: May 22, 2014

(54) METHOD OF TREATING INFLAMMATORY DISEASES USING ADENOSINE 2B RECEPTOR ANTAGONISTS

(71) Applicants: Michael W. Burnet, Tubingen (DE); Thomas Stohr, Mol (BE); Martin Donsbach, Bruhl (DE)

(72) Inventors: Michael W. Burnet, Tubingen (DE); Thomas Stohr, Mol (BE); Martin Donsbach, Bruhl (DE)

(21) Appl. No.: 14/077,211

(22) Filed: Nov. 11, 2013

Related U.S. Application Data
(60) Provisional application No. 61/724,883, filed on Nov. 9, 2012.

(51) Int. Cl. A61K 31/522

(52) U.S. Cl. CPC A61K 31/522
USPC 514/252.16; 514/263.22; 514/263.35; 514/263.34

(57) ABSTRACT

A method of treating inflammatory diseases with Adenosine 2B receptor antagonists in particular with xanthine derived inhibitors. The method involves supplying a therapeutically active amount of the compounds of FIG. 1 and in particular compound 1.

### RELATED APPLICATIONS

This application claims the benefit of and priority to U.S. Provisional Patent Application No. 61/724,883, the entire contents of which are incorporated herein by reference.

### TECHNICAL FIELD

The subject matter described herein relates to a method of treating inflammatory diseases with Adenosine 2B receptor antagonists in particular with xanthine derived inhibitors.

### BACKGROUND

The methods and compositions to be described relate to Adenosine 2B receptor antagonists and their use to treat inflammatory diseases, disorders, and conditions: Adenosine is a ubiquitous signalling agent in mammalian biology and there are, accordingly, mechanisms that allow spatial and temporal specification of adenosine signals. These include a variety of receptor isoforms (A1, A2A, A2B, A3) with varying functions from the class of purine and pyrimidine receptors. A2B receptors are, in particular, expressed in cells of the immune system such as mast cells and are activated in processes like mast cell degranulation. A2B has a moderate affinity for adenosine in the range of 1 μM which is low enough to provide some scope for displacement by a synthetic ligand, however, adenosine is itself relatively abundant being derived from successive dephosphorylation of ATP which is, itself present at mM intracellular levels. It appears that adenosine is ordinarily an intracellular metabolite and that its release into the extracellular matrix would be indicative of cell injury. Hence its utility as a signal for an extracellular receptor.

### BRIEF SUMMARY

The following aspects and embodiments thereof described and illustrated below are meant to be exemplary and illustrative, not limiting in scope.

In one aspect, a method for treating an immune disorder is provided, comprising: administering Adenosine 2B receptor antagonists to a subject suffering from an immune disorder in an amount sufficient to modify disease.

In some embodiments, the immune disorder is an autoimmune disease. In particular embodiments, the immune disorder is rheumatoid arthritis. In other particular embodiments, the immune disorder is multiple sclerosis. In still other particular embodiments, the immune disorder is selected from, psoriasis, psoriatic arthritis, Crohn's disease, systemic lupus erythematosus, pulmonary fibrosis, liver inflammation, myocarditis, transplant rejection and atherosclerosis.

In some embodiments, the Adenosine 2B receptor antagonists is administered by injection at a dose that achieves blood levels of about 0.02 micromolar or greater. In a preferred embodiment, the Adenosine 2B receptor antagonist is injected in a depot formulation such that it provides adequate blood levels for 1 to 28 days. In some embodiments, Adenosine 2B receptor antagonists are orally administered.

In some embodiments, the Adenosine 2B receptor antagonists is orally administered at a dose above 500 mg per day to treat acute disease. In other embodiments, the dose is less than 300 mg/day to treat non-acute disease. In certain embodiments, prophylaxis of immune disease is achieved by daily doses below 200 mg, preferably below 100 mg and still more preferably, in the range 10 to 50 mg per day.

### BRIEF DESCRIPTION OF THE DRAWINGS

FIG. 1 shows a generic structure of the xanthine class of compounds and Adenosine 2B receptor antagonists

FIG. 2 shows a structure of the prototypic Adenosine 2B receptor antagonist Compound 1

FIG. 3 illustrates treatment of collagen induced (CIA) arthritis in mice by treatment with Adenosine 2B receptor antagonist Compound 1, at doses of 30 mg/kg (squares) given i.p. once-daily starting when CIA is established. Control mice were treated with saline (circles). Data are the mean arthritis paw thickness, assessed using a calliper in the days following primary immunization and boost.

FIG. 4A and FIG. 4B illustrate prevention of Experimental Autoimmune Encephalomyelitis (EAE) in mice by treatment with Adenosine 2B receptor antagonists, at doses of 15 mg/kg (circles) given i.p. once-daily starting when EAE is first induced. Control mice were treated with PEG/saline (triangles). FIG. 4A shows the mean weight of animals normalised to weight prior to induction of disease. FIG. 4B shows the mean EAE score, assessed using a visual scoring system from 0 to 5 in which 5 represents full paralysis, and 0, normal activity. Data are the mean plotted with the standard error of the mean.

FIG. 5 illustrates the pharmacokinetics of Compound 1 in C57BLK6 mice by either the oral or intravenous route. Compound 1 is detectable only via injection.

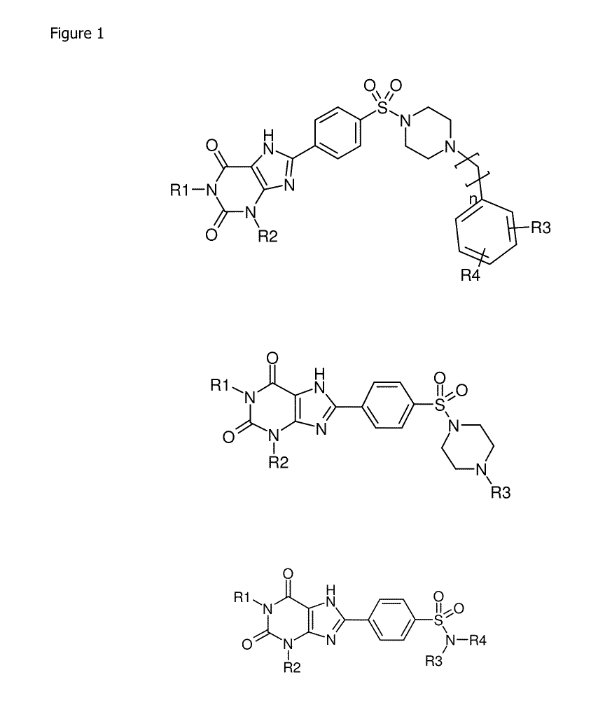

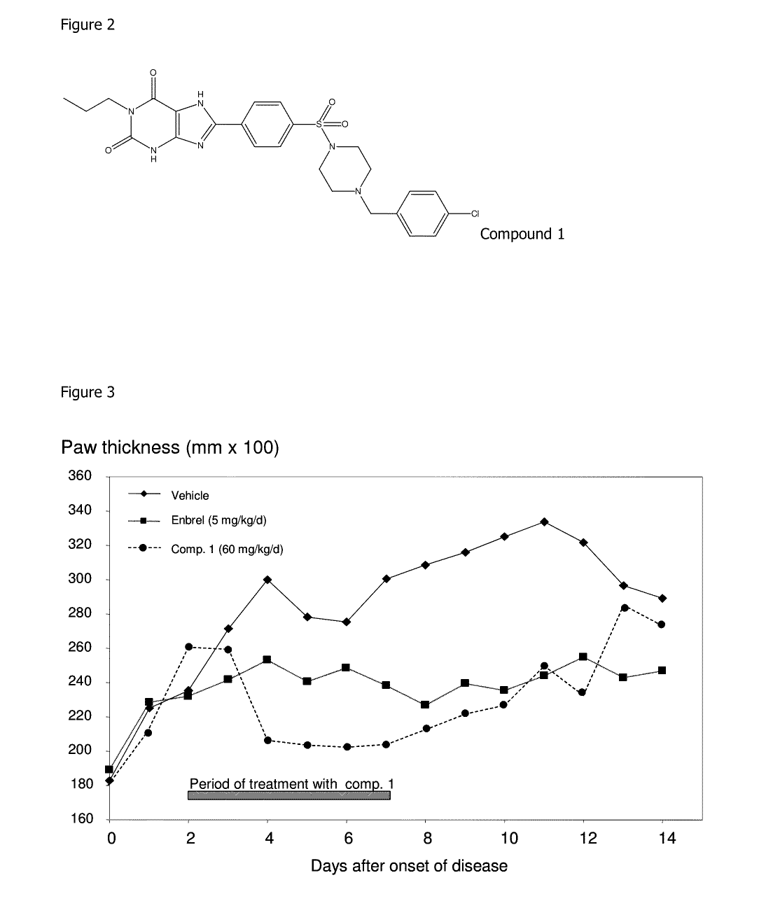

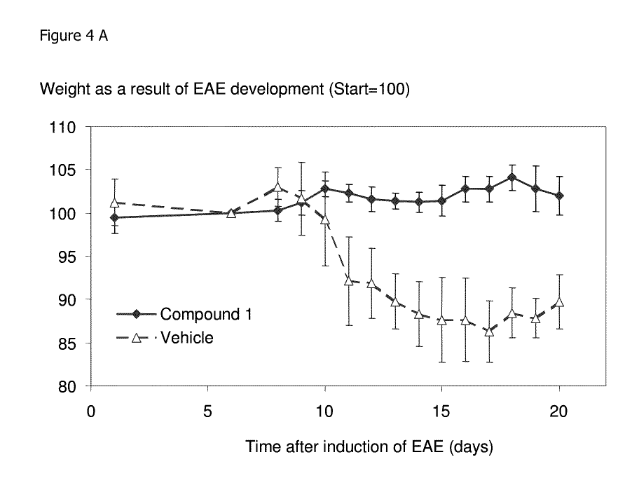

### DETAILED DESCRIPTION

I. Definitions

“Treat” or “treating” means any treatment, including, but not limited to, alleviating symptoms of a disease, disorder, or condition.

“Preventing” refers to inhibiting the initial onset of a pathologic process.

“Therapeutically effective amount” means an amount of a compound that is effective in treating or preventing a particular disorder or condition.

“Pharmaceutically acceptable carrier” is a non-toxic solvent, dispersant, excipient, or other material used in formation of a pharmaceutical composition, i.e., a dosage form capable of administration to a subject or patient.

“Immune disorder” means any disease or pathology that is associated with non-normal function of the immune system such that the immune system is auto reactive, excessively active or otherwise causes a pathological effect on its host that may have an inflammatory component. Examples include rheumatoid arthritis, multiple sclerosis, inflammatory bowel disease, graft rejection, myocarditis, atherosclerosis, asthma, chronic bronchitis and psoriasis. A “selective inhibitor of the adenosine 2 B receptor” is a substance that has a Ki for the receptor of 10 μM or lower and for which the Ki on A2B is at least 10-fold lower than for A1, A2A and A3.

An “adenosine 2 B receptor antagonist” is an inhibitor of the A2B receptor.

A. Overview

In various aspects, methods for treating and preventing inflammatory diseases are described. Such methods include those for inhibiting the initiation of inflammation, inhibiting inflammatory diseases related to mast cells, inhibiting inflammatory diseases involving activated macrophage, and inhibiting inflammatory diseases involving osteoclasts. The methods include administering Adenosine 2B receptor antagonists at a dosage sufficient to selectively inhibit the A2B receptor, thereby modulating the downstream signalling effects of the receptors, causing a beneficial therapeutic affect on a subject/patient.

Adenosine 2B receptor antagonists include inhibitors described in the following publications:

Bormann et al., J. Med. Chem. 2009, 52, 3994-4006, Yan et al., J. Med. Chem. 2006, 49, 4384 4391, Yan and Müller J. Med. Chem. 2004, 47, 1031 1043

Hayella et al., J. Med. Chem. 2002, 45, 1500 1510 along with other analogs of similar activity, all of which are hereby incorporated by reference.

Adenosine 2B receptor antagonists of utility include structures related to the 8-aryl-xanthines substituted with piperidyl-sulfonamides as indicated in the following generic structure.

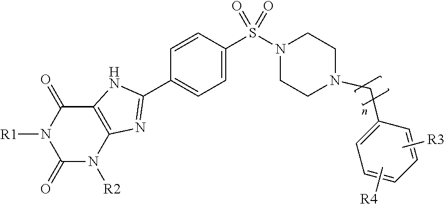

Where R1 and R2 are independently selected from H, Alkyl, branched alkyl, 1-Propin-3-yl, amino alkyl

n is independently selected from 0, 1, 2 or 3,

R3 and R4 are independently selected from H, halogen (F, Cl, Br, I), trifluoromethyl, hydroxyl or alkoxy, methylendioxy.

In a preferred embodiment, R2 is H and R1 is an alkyl group and n is 0, 1 or 2.

In a more preferred embodiment, R2 is H and R1 is propyl, and n is 0

Additional adenosine 2B receptor antagonists of utility include structures related to the 8-aryl-xanthines substituted with piperidyl-sulfonamides as indicated in the following generic structure.

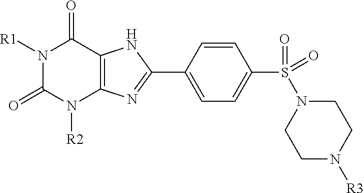

Where R1 and R2 are independently selected from H, Alkyl, branched alkyl, 1-Propin-3-yl, amino alkyl

R3 is selected from H, piperdinyl, pyridinyl, cyclohexyl, cycloheptyl,

Further adenosine 2B receptor antagonists of utility include structures related to the 8-aryl-xanthines substituted with piperidyl-sulfonamides as indicated in the following generic structure.

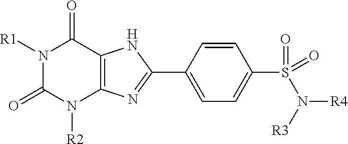

Where R1 and R2 are independently selected from H, Alkyl, branched alkyl, 1-Propin-3-yl, amino alkyl

R3 and R4 are independently selected from H, alkylphenyl, alkyl, branched alkyl, hydroxyalkly, carboxyalkyl and alkylarylether, halo aryl, pyridinyl

In a preferred embodiment, R2 is H and R1 is an alkyl group and n is 0, 1 or 2.

In a more preferred embodiment, R2 is H and R1 is propyl,

The selectivity of these substances at various adenosine receptors is illustrated as follows:

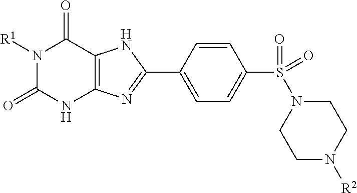

| | | | rA1 | rA2A | hA2B | hA3 |
|---|---|---|---|---|---|---|
| | | | Ki ± SEM | Ki ± SEM | IC50 ± SEM | Ki ± SEM |
| Comp. # | R1 | R2 | [nM] | [nM] | [nM] | [nM] |
| | Propyl- | 3,4-(Methylen- | 79 ± 5 | 590 ± 29 | 24 ± 10 | 4150 |
| | | dioxy)benzyl- | | | | |
| | Propyl- | p-Methoxybenzyl- | 84 ± 19 | 1020 ± 561 | 16 ± 6 | >10000 |
| | Propyl- | p-Chlorbenzyl- | 386 ± 98 | 1730 ± 790 | 4 ± 1 | −10000 |
| | Propyl- | m-Trifluormethylbenzyl- | 273 ± 47 | 1700 ± 217 | 4 ± 0.2 | −10000 |
| 2 | Propyl- | p-Trifluormethylbenzyl- | 463 ± 122 | 1540 ± 271 | 2 ± 1 | >10000 |
| 4 | Propyl- | m-Chlorbenzyl- | 178 ± 38 | 799 ± 358 | 2 ± 0.1 | −10000 |
| 5 | Propyl- | m-Fluorbenzyl- | 111 ± 43 | 782 ± 236 | 3 ± 0.2 | −10000 |
| | Propyl- | Benzyl- | 151 ± 38 | 848 ± 433 | 10 ± 2 | >10000 |
| 1 | Propyl- | p-Chlorphenyl- | >10000 | >10000 | 1 ± 0.4 | >10000 |
| | Propyl- | 2-(Phenyl)ethyl- | >10000 | 154 ± 44.9 | 11 ± 3 | <10000 |
| | Ethyl- | Phenyl- | 142 ± 6 | 514 ± 41 | 4 ± 3 | >10000 |
| | Ethyl- | 3,4-(Methylen- | 54 ± 5 | 332 ± 129 | 5 ± 2 | <10000 |
| | | dioxy)benzyl- | | | | |
| | Ethyl- | p-Methoxybenzyl- | 129 ± 41 | 630 ± 305 | 5 ± 1 | <10000 |
| 3 | Ethyl- | m-Trifluormethylbenzyl- | 509 ± 155 | 499 ± 129 | 2 ± 1 | >10000 |

The methods for synthesising the foregoing compounds are recorded in Bormann et al., J. Med. Chem. 2009, 52, 3994-4006.

Adenosine 2B receptor antagonists for treating rheumatoid arthritis

In studies described herein, it is shown that Adenosine 2B receptor antagonists prevent and treat inflammatory diseases by selectively inhibiting a spectrum of signal transduction pathways central to the pathogenesis of the inflammatory disease. Using collagen-induced arthritis (CIA) in mice as a model of an exemplary inflammatory disease, rheumatoid arthritis (RA), i.p. administration of Adenosine 2B receptor antagonists to mice was shown to be effective in treating the progression of CIA (e.g., Example 1 and FIG. 3) in mice with established clinical arthritis. After the end of treatment, the inhibitory effect was maintained for a period of 2-3 days after which signs began to re-emerge. These data suggest that the activity of the A2B receptor is required for the maintenance of inflammation. Perhaps more importantly, these data suggest that it is possible to rapidly suppress established arthritis in the course of the peak inflammatory potential, a period of CIA that resembles the conditions of an acute arthritic flair.

In further experiments performed in support of the present compositions and methods, the ability of Adenosine 2B receptor antagonists to treat and/or prevent another exemplary inflammatory condition, experimental autoimmune encephalomyelitis (EAE), was evaluated. EAE is a widely used animal model for multiple sclerosis (MS). Mice in which EAE had been induced were treated with Adenosine 2B receptor antagonists once daily i.p. and the severity of the disease was determined using a standard scoring system, described in Example 2. Animals treated with Adenosine 2B receptor antagonist Compound 1 once daily at 15 mg/kg i.p. demonstrated delayed onset such that there were no signs of EAE compared to control mice. Adenosine 2B receptor antagonists likely provided a beneficial therapeutic effect by inhibiting the signalling required to initiate inflammation. While the EAE model is not necessarily able to replicate all aspects of human forms or multiple sclerosis, these data suggest that the Adenosine 2B receptor antagonists are able to exert a general anti-inflammatory action irrespective of the inflamed organ.

Delivery and Formulations

The inhibitors described herein are suitable for once daily injection, however, they are relatively quickly eliminated and thus they are suitable for use in depot and modified release forms as a means to extend their duration of action.

In certain applications, rapid elimination is an advantage in that it provides a reduction in toxicological risk. In this respect, in a preferred embodiment, the inhibitor has a plasma half-life following subcutaneous injection of 8 hours or less in human subjects.

Administration less than every day is also contemplated, for example administration every other day or several times per week or once per week or once every 14 days using formulations designed to have some depot effect. Additionally, intermittent courses of therapy with Adenosine 2B receptor antagonists or are contemplated, for example, treatment for one week then off drug for one week, or treatment for one week then off drug for three weeks, or treatment only during periods of disease flare.

In a preferred embodiment, the Adenosine 2B receptor antagonists is administered parenterally. In one embodiment, substance is provided in a simple propylene glycol suspension for subcutaneous administration. In a further embodiment, the suspension may be incorporated in a matrix such as a poly-lactide or similar biocompatible polymers.

For treatment of disease of the digestive system and the liver, oral administration is contemplated. In inflammatory bowel disease, oral formulations including enterically coated materials are suitable.

Solid dosage forms for oral administration include capsules, tablets, pills, powders, and granules. In such solid dosage forms, the active compound is mixed with at least one inert, pharmaceutically-acceptable excipient or carrier, such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules may be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compound can also be in micro-encapsulated form, if appropriate, with one or more of the above-mentioned excipients.

Liquid dosage forms for oral administration include pharmaceutically-acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents. Suspensions may contain, in addition to the active compounds, suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof. From the foregoing, various additional aspects and embodiments of the present compositions and methods will become apparent. The following Examples are provided to illustrate the compositions and methods but are not intended as limiting.

### EXAMPLES

The following examples are illustrative in nature and are in no way intended to be limiting.

### Example 1

Treatment of rheumatoid arthritis using Collagen Induced Arthritis as a model Collagen-induced arthritis studies. CIA in DBA/1 mice was induced by injecting DBA/1 mice with bovine type Il collagen (CII) emulsified in CFA1 followed by boosting 21 days later with CII emulsified in incomplete Freund's adjuvant (IFA). Technical Adenosine 2B receptor antagonist (Compound 1) was suspended in warm PEG 400 at a concentration of 60 mg/mL and diluted 1 in 10 in saline to form an injectable suspension. 30 mg/kg was delivered by i.p. injection once daily, starting following the development of clinical arthritis in treatment experiments. Animals were monitored daily following boost of arthritis, following the emergence of clear signs of disease observed on two consecutive days, animals were allocated randomly to treatment groups. Signs monitored included weight loss, paw thickness, and observed clinical score. Scoring is according to the presence of inflamed joints: 1 point for an inflamed digit, 1 point for a inflamed metatarsus, and 1 point for an inflamed joint above the metatarsus.

Mice treated with Adenosine 2B receptor antagonists displayed significant reductions in the severity of CIA based on reduced paw swelling, erythema and joint rigidity as assessed by the mean visual arthritis score, as shown in FIG. 3. Of particular clinical importance in these data is the fact that the adenosine 2B receptor antagonist was able to reduce an existing inflammation to a normal state. This is in contrast to substances such as methotrexate that function only in prophylaxis.

### Example 2

### Method of Treating Multiple Sclerosis, the Effect of Adenosine 2B Receptor Antagonists in EAE

Adenosine 2B receptor antagonist compound 1 was tested for its ability to prevent and treat experimental autoimmune encephalomyelitis (EAE), a mouse model of multiple sclerosis (MS). EAE was induced in C57B/6 mice by subcutaneous immunization with 100 ug/mouse myelin oligodendrocyte glycoprotein (MOG) peptide 35-55 emulsified in compete Freund's adjuvant (CFA) containing 2 mg/ml heat-killed mycobacterium tuberculosis H37Ra (Hooke Laboratories). As part of the induction protocol, mice were also injected intraperitoneally on the day of immunization and 48 hours later with 0.1 ml of 4 [mu]g/mL Bordetella pertusis toxin. Severity of EAE was determined daily based on a standard scoring system: 1, tail weakness or paralysis; 2, hind leg weakness; 3, hind limb paralysis; 4, forelimb weakness; and 5, moribund animals or death. Mice treated with 15 mg/kg Adenosine 2B receptor antagonists once daily demonstrated a delay in the onset of EAE compared to the vehicle control mice.

These data demonstrate that the Adenosine 2B receptor antagonists are also efficacious in treating a rodent model of multiple sclerosis.

### Example 3

### Method of Preparing an Injectable Formulation of an Adenosine 2B Receptor Antagonist

Adenosine 2B receptor antagonist compound 1 (30 mg) was suspended in 1 mL of warm (50 C) propylene glycol and vigourously ground using either a mortar (prewarmed) or a bead mill The resulting homogenate was further mixed with 1 mL of saline containing 1 w/V Tween 80 and vigorously mixed. The resulting suspension was then diluted to 10 mL in saline.

### Example 4

### Method of Preparing an Injectable Formulation of an Adenosine 2B Receptor Antagonist

Polylactide co-glycolide was dissolved in N-methyl pyrollidone triacetin to a concentration of 15% W/V according to Madhu et al, 2009. Adenosine 2B receptor antagonist compound 1 (100 mg) was suspended in 1 mL of warm (60 C) polylactideglycolide and homogenised using a bead mill The preparation is tested for its ability to prevent and treat disease as in the earlier examples.

### Example 5

### Pharmacokinetics of Compound 1

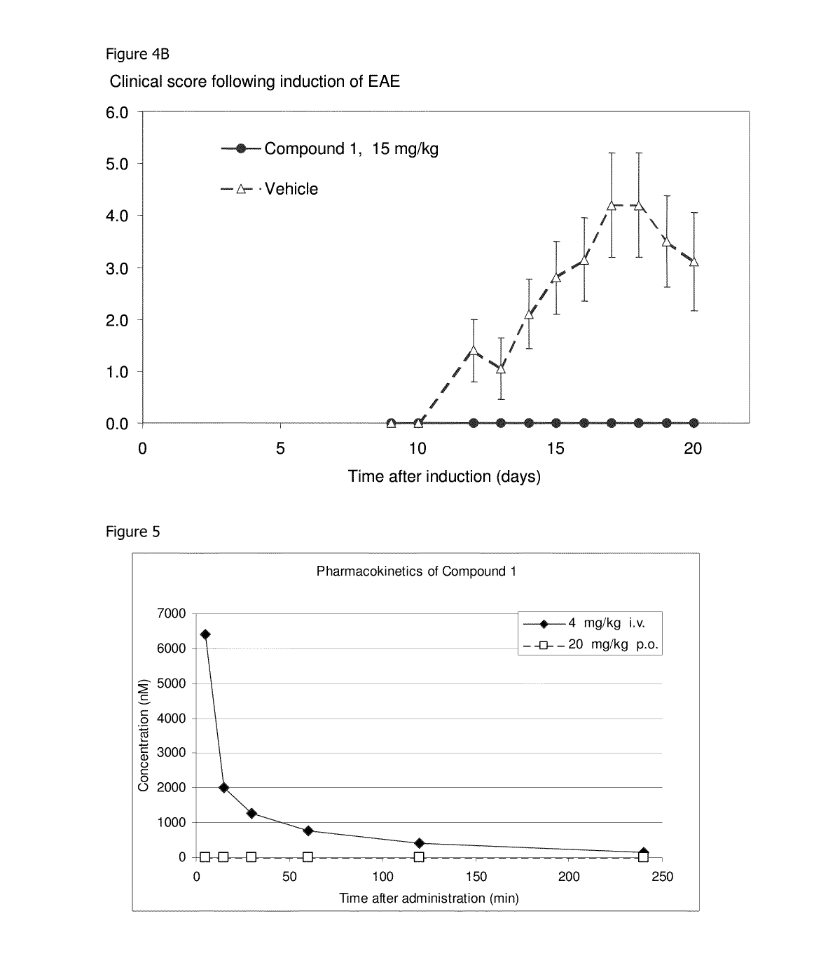

Adenosine 2B receptor antagonist compound 1 was prepared in either the 1% methyl cellulose in water, 0.2% Tween 80 at 5 mg/ml, or in mouse serum 0.8 mg/mL. The cellulose formulation was administered to C57BLK6 mice orally. The serum suspension/solution was administered intravenously. At various times after administration, small samples of blood ca. 20 μL were taken and analysed for compound 1. The results are recorded in FIG. 5. These data show that compound 1 is not orally available, but is has a terminal half life in the range of 1 to 2 h after i.v. application.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### CITED NON-PATENT DOCUMENTS

Bormann et al., J. Med. Chem. 2009, 52, 3994-4006

Yan et al., J. Med. Chem. 2006, 49, 4384 4391

Yan and Müller J. Med. Chem. 2004, 47, 1031 1043

Hayella et al., J. Med. Chem. 2002, 45, 1500 1510

Madhu et al., International Journal of Pharmacy and Pharmaceutical Sciences, Vol. 1 Supp. 1, November-December 2009

What is claimed is:

1. A method of treating an immune disorder, comprising administering to a subject in need thereof an effective amount of a compound that is a selective inhibitor of the adenosine 2 B receptor.

2. The method of claim 1 in which the inhibitor is an 8-aryl-xanthine substituted with a piperidyl-sulfonamide

3. The method of claim 2 in which the inhibitor is 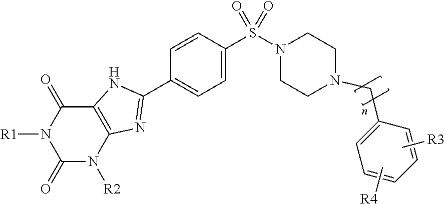
Where R1 and R2 are independently selected from H, Alkyl, branched alkyl, 1-Propin-3-yl, amino alkyl
n is independently selected from 0, 1, 2 or 3,
R3 and R4 are independently selected from H, halogen (F, Cl, Br, I), trifluoromethyl, hydroxyl or alkoxy, methylendioxy

4. The method of claim 2 in which R2 is H and R1 is an alkyl group and n is 0, 1 or 2.

5. The method of claim 2 in which R2 is H and R1 is propyl, and n is 0

6. The method of claim 2 in which the inhibitor is compound 1

7. The method of claim 2 in which the immune disorder is selected from rheumatoid arthritis, multiple sclerosis, inflammatory bowel disease, graft rejection, myocarditis, atherosclerosis, asthma, liver inflammation, type II diabetes, chronic bronchitis and psoriasis.

8. The method of claim 2 in which the immune disorder is selected from rheumatoid arthritis, multiple sclerosis, inflammatory bowel disease, and atherosclerosis.

9. The method of preparing a medicament for treatment of a patient in need thereof comprising mixing the inhibitor of claim 2 with appropriate solvents and excipients.

10. A method of preparing a medicament for treatment of a patient in need thereof comprising mixing compound 1 with appropriate solvents and excipients including propylene glycol, polylactide glycolide, N methyl pyrolidone, PEG and surfactants including cremphores, Tweens and triacetin.

11. A method of preparing a medicament for treatment of a patient in need thereof comprising mixing compound 1 with appropriate solvents and excipients for sub-cutaneous injection.

12. The method of claim 2 in which the inhibitor is 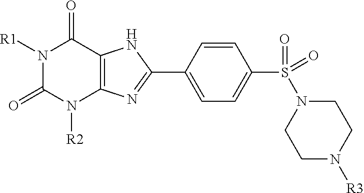
Where R1 and R2 are independently selected from H, Alkyl, branched alkyl, 1-Propin-3-yl, amino alkyl and R3 is selected from H, piperdinyl, pyridinyl, cyclohexyl, cycloheptyl,

13. The method of claim 2 in which the inhibitor is 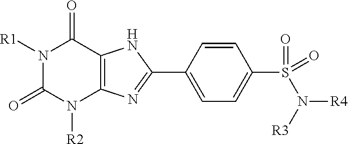
Where R1 and R2 are independently selected from H, Alkyl, branched alkyl, 1-Propin-3-yl, amino alkyl
R3 and R4 are independently selected from H, alkyl phenyl, alkyl, branched alkyl, hydroxyalkly, carboxyalkyl and alkylarylether, halo aryl, pyridinyl

14. The method of claim 12 in which the immune disorder is selected from rheumatoid arthritis, multiple sclerosis, inflammatory bowel disease, graft rejection, myocarditis, atherosclerosis, asthma, liver inflammation, type II diabetes, chronic bronchitis and psoriasis.

15. The method of claim 12 in which the immune disorder is selected from rheumatoid arthritis, multiple sclerosis, inflammatory bowel disease, and atherosclerosis.

16. The method of preparing a medicament for treatment of a patient in need thereof comprising mixing the inhibitor of claim 12 with appropriate solvents and excipients.

17. The method of claim 13 in which the immune disorder is selected from rheumatoid arthritis, multiple sclerosis, inflammatory bowel disease, graft rejection, myocarditis, atherosclerosis, asthma, liver inflammation, type II diabetes, chronic bronchitis and psoriasis.

18. The method of claim 13 in which the immune disorder is selected from rheumatoid arthritis, multiple sclerosis, inflammatory bowel disease, and atherosclerosis.

19. The method of preparing a medicament for treatment of a patient in need thereof comprising mixing the inhibitor of claim 13 with appropriate solvents and excipients.
